(19) United States
(12) Patent Application Publication
(10) Pub. No.: US 2025/0213397 A1
(43) Pub. Date: Jul. 3, 2025

(54) RE-USABLE CHASSIS WITH PARTICULAR FRAME FOR USE WITH DISPOSABLE ABSORBENT PAD

(71) Applicant: W. PELZ GMBH & CO KG, Wahlstedt (DE)

(72) Inventors: Henning RÖTTGER, Kaltenkirchen (DE); Matthias WALLERT, Bad Segeberg (DE)

(21) Appl. No.: 18/850,730

(22) PCT Filed: Mar. 27, 2023
(86) PCT No.: PCT/EP2023/057846

§ 371 Date: Sep. 25, 2024

(30) Foreign Application Priority Data
Mar. 25, 2022 (EP) 22164271.3
Aug. 26, 2022 (EP) 22192264.4

(51) Int. Cl. A61F 13/505; A61F 13/15; A61F 13/56; A61F 13/84

(52) U.S. Cl. CPC A61F 13/505; A61F 13/15203; A61F 13/5638; A61F 13/84; A61F 13/8405; A61F 2013/15276; A61F 2013/15292; A61F 2013/15487; A61F 2013/15552; A61F 2013/8414

(57) ABSTRACT

The present invention relates to a reusable hygiene article chassis comprising a pocket for receiving a replaceable absorbent pad. The chassis comprises a liquid intake zone which is particularly adapted to receive bodily exudates and further transfer these to the replaceable absorbent pad.

### FIELD OF THE INVENTION

The present invention relates to a reusable hygiene article chassis comprising a pocket for receiving a replaceable absorbent pad. The chassis comprises a liquid intake zone which is particularly adapted to receive bodily exudates and further transfer these to the absorbent pad.

### BACKGROUND

Apart from washable cloth diapers, two-piece diaper systems comprising a reusable and washable outer layer and a disposable insert have long been used. Over the last decades, disposable diapers have significantly improved both ease of application and liquid handling performance, which further led to healthier skin of a wearer even at extended wear times, not only for babies but also for incontinent adults as well as for menstruating women. Especially for the latter two categories, absorbent pads are broadly used for lower liquid handling requirements, such as light to medium incontinence or lower menstrual flow conditions, whereby such pads are typically inserted into regular underwear, optionally attached thereto by releasable adhesive, or—mostly in hospitals or care facilities—elasticated pants. In particular for mobile people, discreetness during wear times and ease of replacement become highly relevant. However, with an increased environmental awareness, disposable products have come under scrutiny for using non-renewable resources and/or for an increase carbon footprint in the overall lifecycle, including treatment of the used products.

It is also known to design products in particular for menstrual products or very light incontinence products, being washable with a slightly increased absorbent capacity in the crotch region so as to accommodate small amounts of exudates. Such products may comprise a hydrophilic wad of fibers in the crotch region, optionally in combination with a thin protective coating or film to reduce the risk of soiling of the clothing. Such products may be laundered and reused many times, and as such also allow the use of more expensive materials, such as textile extensible materials, such as knitted cotton, as well known from conventional underwear, or elastic materials, such as rubber, preferably synthetic rubber, or synthetic elastic materials, known as LYCRA™ and the like.

However, the use of such products is limited for several reasons: First, de facto the product has to be washed, at least coarsely rinsed, and dried just after being used. Otherwise, if left wet, not only unpleasant smell may develop upon wet storage, but also bacteria may develop, that will require even harsher washing conditions. To avoid bacterial growth, it is quite common to use textiles treated with biocides for such washable products having the downside of potentially having a negative impact on skin health by destroying the natural bacterial climate of the skin.

Furthermore, biocides also have a negative impact on the environment if they partly get washed off during washing of the product or after disposal.

Further, other additives that are susceptible to be removed during washing, or a least reduced in their efficiency, such as the mentioned bacterial growth inhibitors, or odor reducing agents, or odor masking agents, cannot be employed effectively. This is particularly relevant for superabsorbent materials, as are well-known for increasing the absorbent capacity beyond the one of the hydrophilic waddings, which should not exhibit high liquid absorbency, as otherwise also the drying may become inefficient. Such superabsorbent materials not only dry very slowly but may even lose their absorbency properties upon washing.

Even further, at away-from-home activities, the same difficulties for changing a product arise as described for one-piece articles in the above, further aggravated by the need to stash the loaded product away until being home again.

Also, with regard to environmental compatibility, the re-usable and washable products require an increased use of energy (e.g., for operating equipment, heating laundry water, and treating wastewater) and chemicals (e.g., detergents and water treatment agents), such that a comparison of washable and fully disposable products with regard to “environmental compatibility” is complicated, and undisputed conclusions either way do not yet appear to exist.

In view of the concerns set forth above, it would be advantageous if a wearable absorbent article were available that provides advantages afforded by both disposable and reusable diapers, while reducing the respective disadvantages of these alternatives. It also would be advantageous if a construction were provided that could simplify manufacturing processes and/or reduce costs.

Henceforth several approaches are known to improve usefulness, costs, and environmental compatibility with systems that combine a more efficient disposable absorbent core with a functional reusable chassis.

U.S. Pat. No. 6,142,983 discloses a disposable absorbent article with an outer cover, an absorbent chassis and an absorbent support member which is configured to maintain the absorbent chassis in close contact with the wearer's body in use. US20060247599 refers to an absorbent garment having a garment-like outer shell and an absorbent assembly positioned therein. WO1995010992A1 discloses a diaper assembly including a washable over-garment and a reusable moisture proof sheet positioned on the inner surface side of the garment.

U.S. Pat. No. 3,489,149 (Larson) describes a re-usable and washable panty for menstrual wear having in the crotch portion a pocket for an optional absorbent insert or pad, wherein the user-oriented portion of the pocket is formed from a first hydrophobic, yet moisture-passing layer for improved skin dryness oriented towards the wearer and a hydrophilic layer oriented towards the insert, if present, or the panty material, which has some tendency to retain moisture for the case when not used in combination with an insert pad. The insert may be disposable.

US2021/0100698 (P&G, Langdon) describes a unitary or two-piece article, wherein the chassis portions exhibit particular stretch properties so as to allow good adaptation to body contours, especially in the crotch region.

In EP2376046, a wearable absorbent article to be worn by a wearer about the lower torso is described, comprising a re-useable chassis adapted to receive an absorbent insert therein. The chassis comprises insert fastener components and the absorbent insert comprises a pair of elasticized standing cuffs.

Along these developments, the commercially available PAMPERS™ Hybrid design provides baby diapers with a reusable chassis and a disposable absorbent core—just as at the beginning of diapering, but with more modern materials, especially superabsorbent materials.

Co-pending application EP22164271 (Pelz, unpublished) describes a system with an article chassis that is adapted to receive an absorbent core on its outer surface, so as to ease replacing of the core upon loading, addressing improved positioning but also exchange of a loaded absorbent core.

However, these current approaches fall short of a balancing user friendliness, functionality and minimizing the amount of disposed material. Fully disposable articles may satisfy user friendliness and functionality very well, but do not address the increased dislike versus waste generation. Also, customization, i.e., adapting properties of an article to differing requirements or preferences of a user, is difficult to satisfy with such articles as the choice of materials used to design the product is restricted by material cost which needs to be limited for disposable products.

Fully reusable—hence washable—systems have either very limited absorbent capacity or require high amounts of absorbent material, contributing to bulkiness and discomfort. Furthermore, the choice of materials is limited to materials suitable for the washing step. Especially superabsorbent materials as well as additives controlling pH for skin-friendliness and odor controlling/absorbing additives are mostly not compatible to the washing process due to their interaction with water and detergents.

The present invention addresses the need for products that satisfy the desire for minimal disposable material together with good performance even at higher liquid loads and with customizable properties and functionalities that are incompatible with washing requirements.

### SUMMARY

In a first aspect, the present invention is re-usable article chassis for being worn on the lower torso of a wearer, and in a second aspect a hygiene article system, comprising such a re-usable article chassis and an absorbent pad.

The article chassis exhibits a user-oriented surface and an opposite outer surface, and comprises at least one liquid intake zone adapted to cover a region of the exudate releasing body openings, comprising chassis fluid handling aid material, which exhibits, when tested on the surface adapted to be oriented towards the user
a centrifuge capacity of
   less than about 10.0 g/g or less than about 5.0 g/g or less than about 3.0 g/g,
   and optionally of more than about 0.5 g/g, or more than about 1.0 g/g,
   when tested according to the “Absorbent Capacity—Centrifuge Capacity” Test;
“Liquid Flow Rate” of more than about 0.4 g/min, preferably of more than about 0.6 g/m, more preferably of more than about 0.8 g/min, when tested according to the “Liquid Flow Resistance Test”, as described herein.

The article further comprises a main chassis region preferably circumscribing the at least one liquid intake zone and optionally a transition region, connecting the intake zone and the main chassis region, and optionally a topsheet material covering the intake zone. The article comprises at least one pocket covering at least a portion of the at least one intake zone, comprising a pocket opening adapted for receiving and positioning an absorbent pad in liquid contact with the liquid intake zone. The pocket may be accessible for a user in a pre-use configuration and in an in-use configuration.

Optionally, the main chassis, the intake zone(s) and the transition region, if present, consist essentially of materials and construction elements that are washable at at least 40° C., preferably at at least 60° C., more preferably at at least 90° C. for at least 30 times, preferably at least 60 times. A re-usable article chassis may further comprise one or more elements selected from the group consisting of
a) leg cuffs, preferably at an extension from base to its opposite end of more than 1 cm, preferably more than 2 cm, more preferably more than 3 cm;
b) openable side closure elements that are in a closed configuration;
c) comprising materials exhibiting a Moisture Vapor Transmission Rate (MVTR) of at least 5000 g/m²/24 h, preferably more than 8000 g/m²/24 h when tested according to “Water Vapor Transmission Rate by the Principle of Measuring the Time to Increase Humidity Lyssy/EDANA,” Part 2 NWSP 070.6.R0 (15);
d) comprising materials exhibiting an air permeability of at least 0.2 m³/(m²*s), preferably more than 1 m³/(m²*s) when tested according to ASTM D 737-18 measured at 125 Pa with a surface area of 20 cm².
e) at least one of the main chassis and the transition region comprises material exhibiting a vertical capillary wicking height of less than 10 mm, preferably less than 5 mm, more preferably being essentially non-wicking, according to AATCC 197, 2018 Edition. 2018—Test Method for Vertical Wicking of Textiles, optionally being water repellant or being liquid impermeable;
f) a circumferential extensibility at least in a waist region of at least 10%, preferably 50% and more preferably 100% of the unstretched circumference;
g) a circumferentially seamless material at least in the waist region of the chassis article, optionally a seamless circular knit material;
h) the one or more intake zone(s) comprise(s) chassis fluid handling aid materials that satisfy one or more of the properties selected from the group consisting of
h1) “Horizontal fluid transfer”
   after 5 mins more than about 18 g, preferably more than about 20 g, more preferably more than about 22 g;
   after 10 mins more than about 30 g, preferably more than about 34 g, more preferably more than about 37 g;
   after 20 mins more than about 68 g, preferably more than about 73 g, more preferably more than about 78 g;
h2) “Vertical fluid transfer”
   after 1 min of more than about 4.0 g, preferably more than about 5.0 g, more preferably more than about 5.5 g;
   after 3 mins of more than about 8.0 g, preferably more than about 9.0 g, more preferably more than about 10.0 g;
   after 15 mins of more than about 18.0 g;
h3) “Vertical Wicking Height” of more than about 19 cm, preferably more than about 20 cm, more preferably more than about 22 cm;
h4) Vertical Fluid Uptake”
   at 3 mins of more than about 2.0 g, preferably of more than about 5.5 g;
   at 5 mins of more than about 6.0 g,
whereby all properties are described by parameter determined according to the tests as described herein;
i) the pocket comprises material at the surface opposite to the intake layer which is liquid impermeable;
j) the materials of the main chassis region and an intake region exhibit a difference of at least 10% of
   repeated liquid strikethrough time (NWSP 070.7 R0 (15));
   wetback after repeated strikethrough time (NWSP 070.8 R0 (15));
   Vertical capillary wicking for 0.9% NaCl solution according to AATCC 197, 2018.

In the second aspect, the present invention is a hygiene article system, which comprises such a re-usable article chassis and an absorbent pad, which comprises
a upper core cover sheet with a wearer-oriented surface;
an opposite surface with a lower core cover sheet:
an absorbent core in between comprising absorbent pad absorbent material;
optionally comprising a pad fluid handling aid;
   and is adapted to be positioned in the pocket of the re-usable chassis in registry with at least a portion of the intake zone of the article chassis and the upper core cover sheet is oriented towards the user.

Such a hygiene article system may further comprise a handling aid for easing application and/or removal of the absorbent pad, as may be a stiffening member, adapted to be used for multiple applications of absorbent pads. Alternatively or additionally, the handling aid may be integral with the absorbent pad and preferably comprises one or more elements selected from the group consisting of
k) a tab or a handle as handling aid
   k1) as an extension of a peripheral region of the pad; or
   k2) attached to the outer surface of the pad;
l) a finger pouch on its outer surface as handling aid.

The absorbent pad of a hygiene article system may further comprise one or more of the elements selected from the group consisting of
m) a removable cover;
n) cellulosic pulp exhibiting a pH of less than 6, preferably less than 5.5 according to the “pH measurement test of cellulose”, as described herein;
o) an odor reducing, adsorbing or controlling agent;
p) an odor masking agent;
q) a bacterial growth inhibitor;
r) an inhibitor of the degradation of urea to ammonia and carbon dioxide;
s) absorbent material, optionally superabsorbent material such that the absorbent pad exhibits an absorbent capacity of more than 10 ml, preferably more than 50 ml or more preferably of more than 100 ml of an aqueous 0.9% NaCl solution;
t) absorbent material, optionally superabsorbent material and optionally an acquisition aid, the article exhibiting a rewet value of at less than 10 g, preferred less than 5 g and most preferred less than 1 g when submitted to the “Adult Incontinence Article: Rate of acquisition and Re-Wet test NWSP 070.9 RI (15)”.

The absorbent pad may further exhibit one or more features selected from the group consisting of
u) a urine related Acquisition value of less than 35 see, when submitted to the Urine related Acquisition and Rewet Determination Test PA07/03 (3×30 ml);
v) a urine related Rewet value of less than 15 g, preferably less than 10 g, and more preferably of less than 5 g, and most preferably of less than 1 g, when submitted to the Urine related Acquisition and Rewet Determination Test PA07/03;
w) a menses related Acquisition value of at least less than 20 see when submitted to the Menses related Acquisition and Rewet Determination Test PA 05/02;
x) a menses related Rewet value of less than 5 g when submitted to the Menses related Acquisition and Rewet Determination Test PA 05/01;
y) a surface pH value of less than about 7.0, preferably less than about 5.5, more preferably of less than about 5, when tested according to the surface pH measurement test, as described herein.

Preferably, the absorbent pad exhibits a size smaller than the fluid intake zone of the article chassis, preferably covering less than 90%, preferably less than 70%, more preferably less than 50% of the fluid intake zone, optionally having a liquid permeable lower core cover sheet material.

The hygiene article chassis may comprise a chassis fluid handling aid material, positioned
in direct contact with the liquid intake zone of the article chassis, and/or
towards the opposite surface of the pocket of the article chassis, whereby the lower core cover sheet is liquid permeable.

Also, the hygiene article chassis may comprise
a first chassis fluid handling aid material, positioned in direct contact with the user oriented liquid intake zone of the article chassis and a second chassis fluid handling aid material positioned towards the opposite surface of the pocket of the article chassis, whereby the first chassis fluid handling aid material exhibits
   a “Vertical Wicking Height” or
   a “Vertical Fluid Uptake”
this is higher than the respective value for the second fluid handling aid, when tested according to the test described herein.

### BRIEF DESCRIPTION OF THE FIGURES

FIGS. 1A and B depict specific elements of absorbent systems according to the present invention.

FIG. 2A to D depict specific elements of an absorbent pad according to the present invention.

FIG. 3A to C depict specific combinations of a chassis and an absorbent pad according to the present invention

FIG. 4A to C depict specific executions of a combination of a chassis and an absorbent pad according to the present invention.

The figures are schematically only, and not to scale. Same numerals refer to same or equivalent features or elements, single (') or multiple (“, '”, . . . ) apostrophes indicate duplicate features, such a left and right or front and back, etc.

### DETAILED DESCRIPTION

Within the present context, absorbent hygiene articles are intended to be worn on the lower torso of a human wearer of any age, adapted to receive bodily exudates, especially liquid exudates, such as urine, menses, as are released in the vaginal region, or from the penis, towards the “intake zone” of the article positioned adjacent to the exudate releasing organ.

Generally, absorbent hygiene articles comprise an “absorbent core” for handling the bodily fluids, i.e., acquiring, distributing and storing these therein, and a “chassis” for holding the absorbent core in place on the body of a wearer. Absorbent articles may be donned and doffed by the user him- or herself, or by a care-taker, such as a parent for babies.

Hygiene articles can be classified as
“pants style” articles, which enclose the waist of the wearer firmly connected like a belt and may be executed circumferentially seamless, but which may include side seams, which further may comprise means for tearing these open for easy doffing. Typically, such articles are stretchable or at least extensible for good fit and for easing donning,
or “taped diapers” that comprise fastening means for connecting the front and rear parts around the waist at donning, though these may also be pre-closed during manufacturing, such that they have a pants like appearance, but are easy to be opened and re-closed, if necessary.

In order to adapt to the complex body contours, the chassis often exhibit “elasticity”, such as induced by elastic elements like rubber, preferably synthetic rubber, strands, including LYCRA™ or similar, but also elastomeric polyolefins, as in films or non-woven. In addition, or alternatively, the chassis may comprise structurally “extensible” materials, such as well-known e.g., from knitted cotton underwear, wherein the knitted yarns are essentially non-elastic, but the knitted structure can readily conform to the body contours, albeit at retractive forces that are typically lower than for elastic materials.

An “absorbent pad” generally consists of an absorbent core between an upper, user-oriented coversheet and a lower, oppositely positioned lower coversheet, and may be held in place on the body by other means, such as a specially adapted removable chassis, or regular underwear, optionally aided by non-permanent adhesives and/or “wings” as may be folded around the crotch portion of the underwear.

The term “permanent” refers to a feature that remains essentially unchanged during the intended use, and as such may be a connection, that can only be disconnected destructively, or a treatment, such as a hydrophobization, that remains during the intended uses, although its effect may be reduced.

A “single piece diaper” comprises an “absorbent core” firmly and permanently connected to a “chassis” by which the absorbent core is held in place on the body of the wearer.

For “two-piece systems”, the absorbent core in an absorbent pad and the chassis are separate or at least can be readily separated, though “pre-combined systems”, where the manufacturer combines the core and chassis removably, may be used. The absorbent core may be in a “replaceable” absorbent pad as can be readily removed and for which a like component or a component providing similar or different functionality can be substituted. The chassis may be similar to the underwear, as well known e.g., from hospital “net-pants” holding any absorbent pad more or less accurately in place. The present invention is directed to such a two-piece system.

The chassis of the absorbent hygiene articles comprise an “inner” or “wearer facing layer”, forming the innermost surface which may be in direct contact with the skin during use, typically formed by a skin contact sheet or “topsheet”, but may also include other elements such as (single or multiple) leakage barrier cuffs or inwardly positioned chassis layers in the waist or hip regions. The absorbent hygiene article also comprises an “opposite” or “garment facing layer”, with elements of the chassis that form the outer surface of the absorbent article, such as the backsheet, the side panels, the waist fasteners, and the like, when present. Within the present context the terms “upper”/“inner” and “lower”/“outer” correspond to “towards the user” and “away from the user”, respectively. The term “major surface” describes surfaces of greatest extent of a generally planar or sheet-like structural element and to distinguish these surfaces from the minor surfaces of the end edges and the side edges, i.e., in an element having a length, a width, and a thickness, the thickness being the smallest of the three dimensions, the major surfaces are those defined by the length and the width and thus having the greatest extent.

Absorbent hygiene articles or elements thereof may be “disposable”, i.e., intended for a single use for being discarded, optionally recycled, in an environmentally compatible and hygienic manner.

Absorbent hygiene articles or elements thereof may be fully “re-usable”, which are adapted to be cleaned many times, typically withstanding at least 30 times, preferably at least 60 times without major deterioration of its properties and functionality, and thus withstand washing with conventional detergents, typically at at least 40° C., preferably at at least 60° C. or more, and even more preferably at at least 90° C. Lower temperatures may be acceptable, if particular cleaning agents are employed, or if the materials of the articles have been particularly treated, such as by permanently incorporated biocides. “Limited re-usability” refers to articles that can be cleaned multiple times, though have a higher wear than the fully re-usable ones, and/or may be discarded like disposable articles after several, e.g., up to ten, use cycles.

Examples for fully re-usable articles are so-called “menstruation pants” that exhibit a low to moderate absorbency as may be achieved by hydrophilic fibers, such as cotton fibers or yarns, at a higher basis weight in, optionally also around, the intake zone.

The above mentioned “net pants” for hospital use typically exhibit a limited re-usability, as they can withstand washing conditions, at least for a few times, but may also be discarded, e.g., if heavily soiled by faeces, and may also be torn open for ease of removal in such cases.

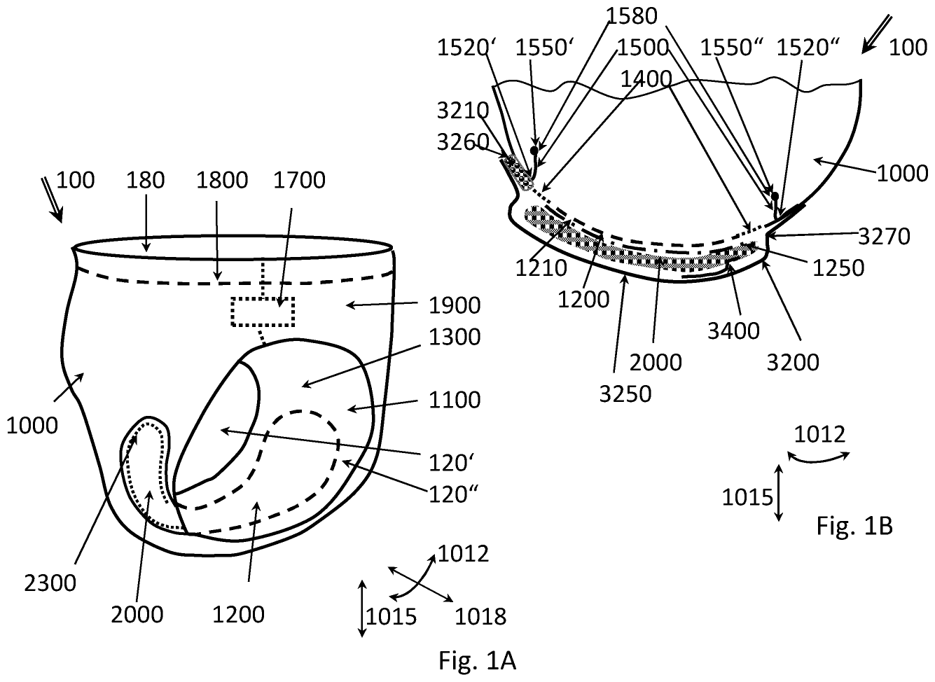

The elements of the present invention as well as their functioning is now explained by referring to FIG. 1 to 4, whereby the shown executions should not be seen limiting. Also, certain features or elements may be used independently from the other elements shown in the figures.

When describing specific properties of materials and/or of elements, reference is made to the “Test Method” section herein below, wherein the determination of the respective parameter by specific test methods is described in detail.

In a first aspect, the present invention is a re-usable article chassis, and in another one two-piece hygiene article system, or kit, 100, comprising such an article chassis and a single use absorbent pad, as exemplarily depicted in FIG. 1 in a shape as may be on a wearer, with a waist opening 180 and leg openings 120′ and 120″, comprising an article chassis 1000 and a separate removable absorbent pad 2000, whereby both are adapted to cooperate with each other, with details thereof being described herein after. Such a system not only offers significantly improved environmental footprint versus a one-piece system, but also allows better handling by a user. Further, such a system also allows to allot specifically functionalities or properties to one part of the system, say the single use absorbent pad, which would not be compatible with the chassis, for example because of the washability requirement, or be this because of cost considerations.

The article chassis 1000 exhibits a width (y-) 1018 and a length (x-) 1012 direction, the first corresponding to a left-right orientation on a user, the second corresponding to a line extending form the front, i.e., navel, through the crotch to the back, i.e., small of the back, of a user. A thickness (z-) direction 1015 extends perpendicularly to both, whereby the terms “upper”/“inner” and “lower”/“outer” correspond to “towards the user” and “away from the user”, respectively.

The skilled person will readily apply such terms also to a chassis and an absorbent pad when detached from a user, such as when manufactured or packaged.

The chassis 1000 is preferably executed in a “pants style”, but may also be in an open diaper style, as indicated in FIG. 1A with dotted lines, whereby the front and rear side portions are connected by closure elements 1700, as fixed by a user or optionally already at manufacturing (“pre-closed”).

The chassis 1000 is a re-usable and washable pants shaped article, with a wearer facing inner layer 1100, which is essentially continuous but comprises a fluid intake zone 1200 and a main chassis region 1300, exhibiting a significant difference of at least one property, e.g., hydrophilicity, porosity, or rewet. As depicted in the figure, the fluid intake zone 1200 may be completely circumscribed by the main chassis region 1300, though it may extend towards the perimeter of the chassis, e.g., laterally in the crotch region. Optionally there may be a transition region 1400, see FIG. 1B, where the materials of the intake zone 1200 and the main chassis region 1300, if different, may be connected, or where the change of property/ies occur/s. Generally, the chassis exhibits superior fit properties, good softness, a nice hand and appearance, such as known from textile, e.g., knitted cotton, underwear.

It is important that the intake zone 1200 comprises a chassis fluid handling aid material that exhibits balanced liquid handling properties, i.e., good acquisition allowing released exudates to penetrate through and away from the wearer, as well as low rewet properties for a dry feel even after loading. Further, the chassis fluid handling aid material shall allow good liquid transfer of the loaded liquids to the absorbent pad and should not exhibit a high absorbent capacity that would detrimentally affect washing and drying. Thus, the intake zone 1200 comprises chassis fluid handling aid materials 1250, such as liquid acquisition or distribution materials, which may improve liquid uptake, but should not compromise rewet properties. Optionally, the intake zone or at least the chassis fluid handling aid material may be covered by a topsheet 1210.

The chassis 1000 comprises a pocket positioned underneath the wearer facing layer 1100 which is adapted to receive an absorbent pad 2000, whereby the application of the absorbent pad 2000 to the chassis 1000 may be made before donning of the combined chassis/absorbent pad system, but can also be readily performed after donning of the chassis without pad and/or when replacing a loaded pad. The chassis comprises a pocket opening for administering the absorbent pad, which may be positioned in the outside of the chassis allowing easy access even after donning, or inside of the chassis.

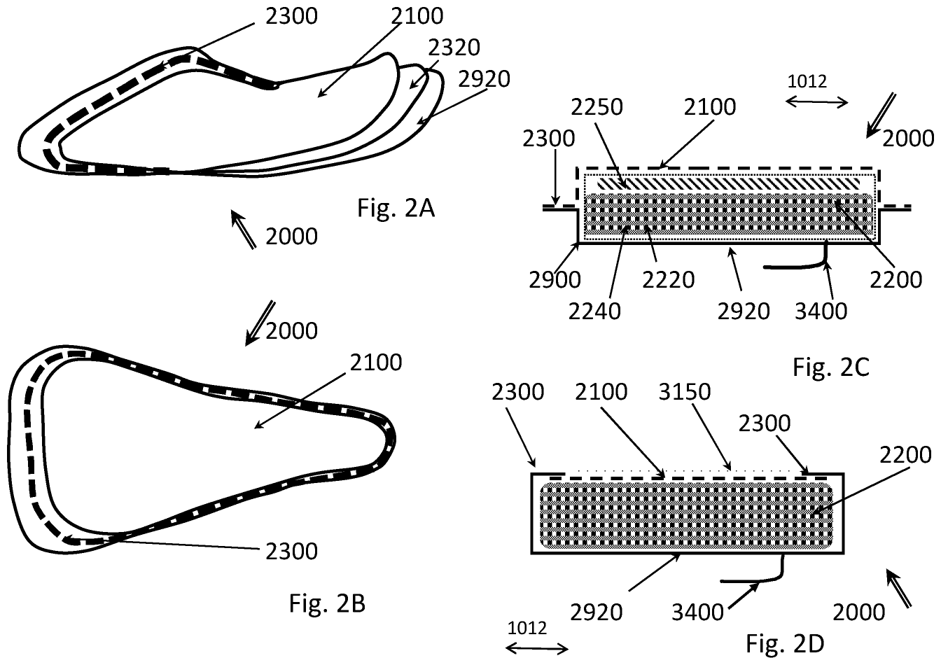

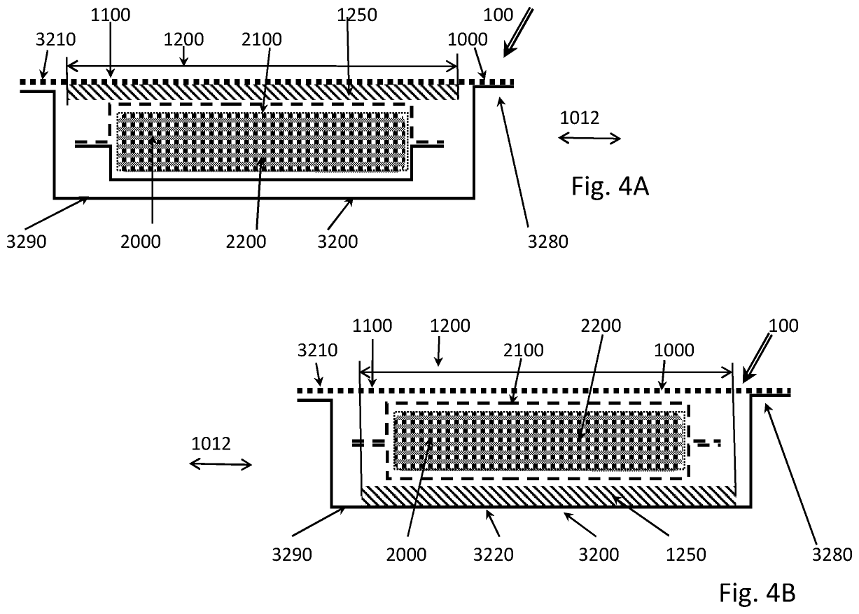

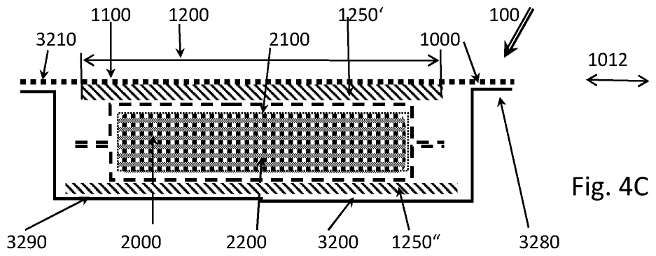

Referring to FIG. 2A to C the absorbent pad 2000 can generally be designed as known in the art, i.e., comprising an absorbent core 2200 with absorbent material, such as pulp 2220 and preferably also superabsorbent material 2240 as ultimate liquid storage material, whilst being thin and soft or pliable for comfortable use, both when dry and loaded. It may comprise further pad liquid handling aids 2250, as liquid acquisition or distribution aids. The absorbent pad should be adapted to cooperate with the intake zone of the chassis. Typically, though not necessarily for all design options, the absorbent pad comprises a liquid barrier 2900 on the garment-oriented side, such as lower core cover sheet 2920, and a fluid permeable upper core cover sheet layer 2100 on the user-oriented surface. However, also the lower core cover sheet may be executed as a liquid permeable material, in particular if the chassis article comprises an additional or alternative chassis liquid handling aid 1250 positioned towards said opposite surface of said pocket of said article chassis, which allows liquid transfer to the absorbent pad side that is remote from the wearer (see FIG. 4 B, C).

The lower core cover sheet and the upper core cover sheet may be connected to each other along the periphery 2300 of the absorbent pad. Optionally the absorbent pad may contain further additives, especially additives that are unsuitable for being added to the chassis, such as odor absorbing or controlling materials, pH controlling materials providing a healthy skin pH or urease blocking agents for preventing release of ammonia, odor reducing or complexing agents, such as cyclodextrins, or odor masking agents, such as fragrances, or biocides or other active ingredients which are not suitable for a laundry process. It is important that the combination of the chassis 1000 and the absorbent pad 2000 is executed such that these are in good liquid contact such that good liquid transfer from the intake zone of the chassis to the absorbent pad is enabled and maintained during the full use period and preferably the chassis 1000 and the pad 2000 are adapted to each other so as to exhibiting
non or minimally hindered liquid transfer from the intake zone 1200 of the chassis towards the ultimate liquid storage in the absorbent core 2200;
minimal rewet, in particular in the intake zone 1200;
good liquid sealing towards the outside of the system 100;
easy application, sustained fixation but also easy removal especially of the absorbent pad 2000 without needing to doff the chassis 1000.

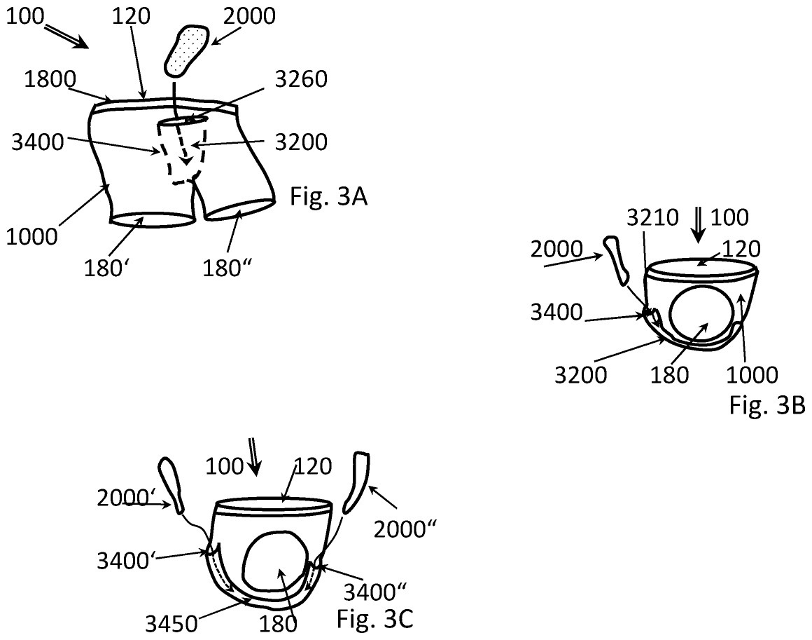

As exemplarily shown in FIG. 1B, the combination may be pockets 3200 of the chassis 2000 with pocket opening 3210 covering a portion of the intake zone 1200, or at least allowing access thereto, adapted to allow positioning of the absorbent pad 2000 therein. The pocket can be executed with three sides being permanently connected to the outer surface 1900 of the chassis, see FIG. 3A. The pocket may be executed as an additional material positioned on the backsheet of the article chassis, covering an opening in the backsheet, thereby allowing the inserted absorbent pad to come into contact with the intake zone of the chassis. The pocket opening may be executed as an opening or slit through the backsheet of the article chassis. Alternatively, the opening may be positioned in the waist region, whereby the topsheet and the backsheet of the chassis are not connected to each other. For particular executions, the pocket opening may also be through the topsheet of the article chassis.

The pocket may comprise attachment elements 3260 for removably closing the pocket.

The outwardly positioned pocket materials 3220 may be liquid impermeable material, such as a plastic film which can be breathable for improved comfort, or film/nonwoven composite, or liquid repellant textiles or liquid permeable materials, depending on the liquid barrier properties of the lower core cover sheet of the absorbent pad.

The material of the pockets may be elastic, such that upon application it is stretched to then apply forces to the pad/chassis interface in the intake zone, thereby enhancing contact and improving liquid transfer, or even further enhance the body contact.

It should be noted that the fluid intake zone may have shapes and sizes that are adapted to the intended use, e.g., being designed for a female application, a male application or a non-gendered application. Accordingly, also the connection elements may be adapted. A pocket design with a slit-type opening positioned towards the front waist of a wearer, see FIG. 3A, may be particularly useful for a male application. For a female design, a pocket with a pocket opening positioned more towards the crotch region may be appropriate, see FIG. 3B, or even a “tunnel pocket”, see FIG. 3C, with pocket openings in the front and in the back of the chassis article, as may be particularly useful, when also the anal region should be covered, optionally by using a secondary absorbent pad.

Having described the hygiene article system with its key elements chassis and absorbent pad in general terms and with a focus on the interaction of the elements, the following will describe particular or exemplary executions and features of these elements, still referring to the FIG. 1 to 4. The skilled person will readily realize that various specific features of the hygiene article chassis and the absorbent pad may be combined for a particular article chassis, or a particular pad, or a particular system comprising a particular article chassis and a particular absorbent pad.

The size and shape of chassis should be adapted to the intended use and cover the respective exudate releasing regions of the wearer, as described in the above. At least for these regions a tighter fit is preferred, like briefs, whilst outside thereof in the main chassis regions a wider fit, such as of “boxer shorts”, is acceptable.

Optionally, the chassis may comprise further leakage barriers 1500, see FIG. 1B shown as a front 1500′ and a rear 1500″ barrier, positioned longitudinally outside of the intake zone 1200, such as well-known from conventional articles as “barrier leg cuffs”. Such leakage barriers are connected at their base 1520′, 1520″ to the inner surface of the chassis, extend at least 1 cm, or 2 cm, or 3 cm away from the base, and may end with an elastic thread 1550′, 1550″ at the end 1580 opposite of the base. Such leakage barriers may also exhibit a primarily longitudinal extension and extend over the full length of the chassis. Such leakage barriers may be made of any liquid impermeable material, preferably hydrophobic nonwoven or textile, more preferably liquid impermeable up to a hydro-head of more than about 10 mm, or more than about 20 mm, or more than about 40 mm when measured according to EDANA NWSP 080.6 R0 (15) “Evaluation of Water Resistance (Hydrostatic Pressure)”.

The chassis should be designed to be readily and easily donned and doffed. It may be executed as a “taped system” that comprises fastening means for connecting the front and rear parts around the waist, which are preferably pre-closed during manufacturing, such that they have a pants like appearance, but are easy to be opened and re-closed, if necessary. In an often-preferred execution, the chassis is designed in a “pants style”, which encloses the waist of the wearer firmly connected like a belt, but which may include means for tearing a side seam open for easy doffing. Also, a seamless articles chassis, such as with a circular knit material, may be very suitable. Typically, such articles are stretchable or at least extensible for good fit and for easing donning.

In order to allow easy donning and also to maintain good adaptation to body contours, suitable materials may exhibit an extensibility or elasticity, of at least about 5%, preferably of more than about 10% or more than about 30% or more than about 50%, relative to the un-extended length.

Preferably, the materials exhibit a low hysteresis, so the material gets back in shape after stretching, e.g., when tested according to the tensile testing NSWP 110.4, and a low degree of “set”, so as to maintain the shape during use or avoid sagging. Preferably, retractive force are balanced so as to maintain the article in its position, even upon loading, without impacting the wearer unduly, such as red-marking would do.

The chassis may also comprise different regions of elasticity and extensibility, for example the main chassis region may exhibit extensibility as may be provided by machine knitted cotton or blends of cotton and elastic synthetic fibers. Typical blend ratios are in the range of 70% to 98% cotton and 2% to 30% elastic fibers. Instead of cotton also blends of cotton and synthetic fibers like polyester (PET) or polyamide (PA) may be used. The chassis article may further comprise an elastic waist band 1800, see FIG. 1A, that may exhibit different elastic properties than the main chassis material. Preferably, the chassis exhibits a circumferential extensibility at least in the waist region of at least 10%, or more than 50%, or more than 100%, relative to the unstretched circumference.

It is highly preferred that the chassis, at least the main chassis region, is “breathable” (air permeable) by exhibiting air permeability values of more than about 0.2 m³/(m²*sec), or more than about 1.0 m³/(m²*sec), when tested according to ASTM D737.18.

Suitable materials for the main chassis may be as follows, without intending to limit the selection:
textile material, such as knitted or woven materials, e.g., double or fine rib or Jersey, as known from conventional cotton or Rayon/Viscose fibers or blends which may contain elastic fibers like Lycra, such as without limitation
   100% knitted cotton, such as used in White sports briefs with double-rip mesh—Original Classics; Product number: 005044-100, of Schiesser GmbH, Germany;
   knitted mix of 95% natural organic cotton and 5% spandex, such as used in Boxer briefs organic cotton dark blue—95/5 Product number 174004-803, of Schiesser GmbH, Germany;
   knitted mix of Polyamide 70% Elastane 30%, such as used in Maxi-Slip Microware white Invisible Soft, Product number: 166916-100 of Schiesser GmbH, Germany;
   knitted Modal 35%, Cotton 35%, Polyamide 20%, Elastane 10%, such as used in Boxer briefs modal Hollow Air technology fine heather aquamarine—Piqué; Product number: 176695-813 of Schiesser GmbH, Germany:
non-woven materials, such as spunbonded non-woven materials, e.g., polypropylene webs with fibers of about 1.7 dTex to about 3.0 dTex; or carded nonwoven materials using blends of fibers which may be bonded by hydroentanglement;

Also vapor permeable film materials may be employed, which may exhibit a vapor permeability of more than 5000 (g/m²/24 h), or more than about 8000 (g/m²/24 h) or more than about 10000 (g/m²/24 h) according to “Water Vapor Transmission Rate by the Principle of Measuring the Time to Increase Humidity Lyssy/EDANA,” Part 2 NWSP 070.6.R0 (15).

Suitable chassis should be washable, i.e., withstand at least one, or at least 10, or at least 30, or at least 60 wash cycles in a conventional household washing machine, e.g., Miele WCA030 WCS-type of Miele & Cie.KG, Germany, at at least 40° C., preferably 60° C., more preferably 90° C. and 1400 rpm spinning speed. Preferably, suitable chassis should be dryer or tumbler compatible, at least under “gentle drying” conditions. Optionally, the chassis materials may be treated to reduce bacteria or odor generating effects.

In the intake zone 1200, the properties should preferably differ from the ones in the main chassis region in at least one property, preferably more than one, that is relevant for liquid handling, which may be selected from the group consisting of
hydrophobicity/hydrophilicity;
porosity or aperturing;
surface topography, such as embossing, such as of bending lines, or channel structures;
material composition, thickness, densities, basis weights;
rewet and/or fluid acquisition performance.

As particular examples, the intake zone may be made by modification of the main chassis material, such as by applying a particular treatment, such as a hydrophobization and/or perforation or a hydrophilization in case of a hydrophobic chassis material, or may be a different material connected in a transition zone 1400 to the main chassis material by conventional means, such as without limitation stitching, gluing, and thermobonding.

The intake zone 1200 should exhibit balanced fluid handling properties of allowing quick acquisition of fluid, but also quick and effective fluid transfer to the absorbent pad 2200. Thus, the materials comprised in the intake zone, especially the intake zone topsheet may exhibit one or more of the following properties:
The material should be hydrophilic according to the Single Load Drop Test with a penetration time of less than 30 sec, preferred 15 sec and most preferred less than 5 sec or even spontaneously penetrating the material.
The “Nonwoven Run Off” values according to EDANA NWSP 080.9.R0 (15) are preferably less than about 50%, or less than about 25% or less than about 10%, or even essentially 0%.
“Air Permeability of Nonwoven Materials” according to ASTM D 737-18 (measured at 125 Pa with a surface of 20 cm²) preferably more than about 0.5 m³/(m²*s), or more than about 1.0 m³/(m²*s) though typically less than about 3.0 m³/(m²*s).
Aperturing, such as by apertures exhibiting a size corresponding to a circle having a diameter of more than about 0.25 mm, or of more than about 0.5 mm, or of more than about 1 mm or even more than about 5 mm, and typically less than about 15 mm or less than about 10 mm, whereby the apertures may also exhibit a three-dimensional funnel-like shape.
“Repeated Liquid Strike-Through Time (Simulated Urine)”, EDANA NWSP 070.7 (3 times 5 ml 0.9% NaCl) of less than 50 s, or less than 25 s, or less than 15 s or less than 10 sec.
“Wetback After Repeated Strike-Through Time (Simulated Urine)” EDANA NWSP 070.8 of less than about 10.0 g, or less than about 5.0 g, or less than about 1 g or even less than about 0.5 g.

A particularly suitable exemplary topsheet material is a 92% polyester/8% Elastane wale/flat knitted web at a basis weight of 210 g/m² at a thickness of 0.85 mm with fibers of approximately 18 μm diameter, available form Ruey Tay Fiber Industry Co. Ltd., Taiwan, under the designation R150411.

It is a particular feature of the present invention that the intake zone 1250 comprises a chassis fluid handling aid material 1250 which possesses particularly balanced fluid handling properties, as are suitably described by the following parameters with their respective determination methods:
“Vertical Wicking Height” and “Vertical Fluid Uptake” reflect the capillary wicking capability of a material when tested according to the “Vertical Wicking Test” as described herein below. The “Vertical fluid uptake” is determined by monitoring the fluid uptake of a material vertically hanging with its lower end immersed into a fluid reservoir overtime, and should be
   at 3 mins more than about 2.0 g, or more than about 5.5 g,
   or at 5 mins more than about 6.0 g.
   The Vertical Wicking Height is the equilibrium height and monitored after 30 mins, and should be
      more than about 19 cm, or more than about 20 cm, or more than about 22 cm.
“Liquid Flow Resistance” reflects the capability of a material to both receive liquid via capillary wicking and release the liquid by gravity according to the “Liquid Flow Resistance Test” as described herein below and resulting in a measure of “Liquid Flow Rate”, which should be more than about 0.4 g/min, or more than about 0.6 g/m, or more than about 0.8 g/min.
“Horizontal fluid transfer” reflects the capability of a material to receive liquid and release the liquid to a standard absorbent core in a horizontal positioning as described herein below in the “Horizontal Liquid Transfer Test”. The material should exhibit
   after 5 mins more than about 18 g, or more than about 20 g, or more than about 22 g;
   after 10 mins more than about 30 g, or more than about 34 g, or more than about 37 g;
   after 20 mins more than about 68 g, or more than about 73 g, or more than about 78 g.
“Vertical fluid transfer” reflects the capability of a material to receive liquid and release the liquid to a standard absorbent core in a vertical positioning as described herein below in the “Vertical Liquid Transfer Test” as described herein below
   after 1 min of more than about 4.0 g, or more than about 5.0 g, or more than about 5.5 g;
   after 3 mins of more than about 8.0 g, or more than about 9.0 g, or more than about 10.0 g;
   after mins of more than about 18.0 g;
“Liquid retention” (centrifuge capacity) of
   less than about 10.0 g/g, or less than about 5.0 g/g, or less than about 2.0 g/g, but more than about 0.5 g/g or more than about 1.0 g/g.
“Liquid Intake Time and Rewet” over a standard absorbent material of
   less than 35 seconds intake time and
   less than 15 g, or less than 10 g, or less than 5 g, or less than 1 g rewet.

Suitable materials for the intake zone may be made selected according to such requirements from a wide choice of material compositions, like nonwoven materials, such as spunbonded, carded, melt-blown webs comprising alone or blended with each other following materials:
hydrophilic synthetic fibers, such as comprising polyolefins, polyester, polyamides, optionally including a surface treatment, optionally a permanent surfactant or including a hydrophilic melt-additive;
natural fibers, such as cellulosic fibers like cotton, hemp, flax, bamboo, or silk;
modified natural fibers such a cellulose based, cotton, hemp, flax, bamboo, with hydrophobic treatment;
regenerated natural fibers, such as cellulose, e.g., viscose, rayon, lyocell based materials, optionally treated, such as hydrophobically treated as know from OLEA™ fibers of Kelheim Fibers, Germany.

Other suitable materials include textile webs, such as knitted, woven, or net structures, of threads or filaments as may be made from materials as also may be used for non-wovens. Also apertured films and film/textile laminates, in particular apertures films exhibiting three dimensionally shaped, funnel-like apertures, optionally combined with a fiber layer on the user-oriented side, may be used.

As indicated in FIG. 1B, in the intake zone 1200, the chassis fluid handling aid material 1250 may form the inner surface layer 1100, or a further material, such as well-known topsheet materials 1210, or may exhibit a composite of z-directionally 1015 arranged sub-layers that exhibit different properties, e.g., the uppermost user-oriented topsheet layer 1210 may exhibit superior softness, skin friendliness or other properties at cost of e.g., fluid uptake speed for higher loads. Then a second or sub-layer may function as the chassis fluid handling aid material 1250. adapted to the absorbency properties of the absorbent pad. When—as indicated in FIGS. 4B and C, a chassis fluid handling aid material 1250 is positioned towards the opposite surface of said pocket of said article chassis, i.e., away from the user relative to the absorbent pad, the inner surface layer should obviously be a separate material, with properties as described above.

Without wishing to be limited it has been found that terry cloth fabrics may suitably satisfy such a requirement, if these are selected according to the above referenced requirements. Without wishing to be bound by the theory, it is believed that the such properties of particular terry cloth fabric stem from the combination of a lofty pile portion, which can readily acquire any fluids, and a denser base portion, enhancing liquid transport.

An exemplary suitable material is a 100% polyester base knit fabric with single sided closed loop piles, with a fiber thickness of about 11 μm, at a basis weight of approximately 260 g/m² and a thickness of 1.7 mm, hereinafter referred to as “Terry cloth-1”, available from Ruey Tay Fibre Industry Co., Ltd, Taiwan under the trade designation 91023FM, whereby the looped side is the user-oriented side during use and testing.

Such an exemplary material has been compared to conventional materials.

A first one, dual side closed loop terry cloth, hereinafter referred to as “MyLily”, has been extracted from a commercially available menstruation pant, currently sold under High Waist—Periodenunterwäsche by MYLILY, Hamburg, Germany, and believed to be constructed from 80% polyester and 20% Nylon, advertised for an in-use capacity of up to 30 ml. For the extracted material the length orientation is maintained throughout the testing.

A second comparative material is a dual side closed loop terry cloth, with fibers at 11 μm diameter of 80% polyester and 20% Nylon, at a basis weight of 250 g/m², and a thickness of 2.8 mm.

A comparison according to the above reference properties according to the Test Methods as described herein below gave the following results (Table 1).

TABLE 1

| | | Terry | | Terry |
|---|---|---|---|---|
| Test | Result units | Cloth-1 | MyLily | Cloth-2 |
| Vertical | [cm] | 22 | 19 | 11 |
| Wicking Height | | | | |
| Vertical Fluid | [g at 3 mins] | 5.7 | 5.4 | 1.5 |
| Uptake | [g at 5 min] | 6.1 | 6.0 | 1.8 |
| Liquid Flow | [g/min] | 0.86 | 0.33 | 0.23 |
| Resistance | | | | |
| Horizontal Fluid | [g at 5 min] | 22.5 | 17.2 | — |
| Transfer | [g at 10 min] | 78.2 | 30.5 | — |
| | [g at 20 min] | 78.2 | 66.8 | — |
| Vertical Fluid | [g at 1 min] | 5.8 | 2.7 | 3.7 |
| Transfer | [g at 3 min] | 10.0 | 6.5 | 7.9 |
| | [g at 15 min] | 18.3 | 15.2 | 18.0 |

Thus, the selected parameters provide an appropriate tool to distinguish inventive materials, and especially the example of Terry-cloth-1, from comparative ones.

The benefits of Terry-cloth-1 was further established by comparison with conventional market incontinence products, namely
Teby, Italy: Menstrual and Incontinence Underpants—Slip—Black
Taynie, Germany: Taynie Herren Slip 500 ml S/M
Essity, Sweden: TENA Silhouette Waschbare Unterwäsche|Classic Größe L

The inventive Sample 1 was made of a composite stack in the order top to bottom of
a topsheet material 92% polyester/8% Elastane wale/flat knitted web at a basis weight of 210 g/m² at a thickness of 0.85 mm with fibers of approximately 18 μm diameter, available from Ruey Tay Fiber Industry Co. Ltd., Taiwan, under the designation R150411, as described in the above;
an upper chassis fluid handling aid material Terry-Cloth-1, as described in the above;
an absorbent structure made of an airlaid material available from Glatfelter, Germany under the trade designation MM460.SMT.CST00.V01.J.NOC, exhibiting
   basis weight of 460 g/m² (NWSP 130.1.R0 (15),
   60 w-% of SAP,
   thickness 1.8 mm (NWSP 120.6.R0 (15),
   calculated density of 0.256 g/m³;
   dry tensile strength—(MD) 48 N/50 mm (NWSP 10.1.R0 (15),
   a “Liquid Retention” of 20 g/g,
   at a width of 46 mm and a length of 200 mm,
enveloped by a hydro-entangled cotton web of 35 g/m², available from Winner Medical, China, under the trade designation PCO-35-N, thus forming the standard absorbent material at a width of 50 mm;
a lower chassis fluid handling aid material Terry-Cloth-1, as described in the above, except that it further comprises a monolithic polyurethane film of approximately 18 g/m².

The comparison was made according to the “intake time and rewet” test as described in the Test method section herein below with the results as shown in Table 2:

TABLE 2

| | | Inventive | | | |
|---|---|---|---|---|---|
| | | Sample 1 | Teby | Taynie | Essity |
| Intake | 1^{st} 30 ml | 12 | 20 | 12 | 24 |
| time [sec] | 2^{nd} 30 ml | 13 | 14 | 12 | 21 |
| | 3^{rd} 30 ml | 12 | Failed (leak) | 11 | Failed (leak) |
| | Total | 37 | | 35 | |
| Rewet [g] | | 10.2 | Failed (leak) | 33.1 | |

Thus, the inventive combination provides a superior combination of intake time and rewet.

Between the intake zone and the main chassis region there may be a transition region 1400, see FIG. 1B, wherein the properties may gradually or stepwise change. The transition zone may also exhibit properties different from the intake zone and the main chassis region, e.g., being executed as a fully hydrophobic barrier between the intake zone with a balanced hydrophobicity and the main chassis region as may be hydrophilic.

An example for a material used for the transition region is a Polypropylene fabric (hydrophobic) e.g., POLYCOLON® available from Omeule OY, Finland.

The various materials of the article chassis may be connected by conventional construction elements that withstand the requirement of multiple wash cycles, such as—without limitation—stitching or thermo-fusion bonding like heat-pressing or ultrasonic bonding. Also, certain adhesives or glues may be employed, if these satisfy skin compatibility (including softness) as well as temperature and washing conditions resistance.

The absorbent pad comprises an upper core cover sheet 2100, a lower core cover sheet 2920, and an absorbent core 2200 (see FIG. 2) z-directionally interposed there between. Optionally, the upper and lower core cover sheet may be unitary, e.g., forming a full core wrap.

The upper core cover sheet is adapted to be in liquid contact with the chassis at least in the intake zone and optionally also in the transition zone, if present. The upper core cover sheet may be a conventional topsheet material as used for absorbent articles, e.g., 10 g/m² spunbonded PP NW, 1.5 to 2.0 dTex. In order to promote the transfer liquid from the fluid handling aid (intermediate storage layer) to the core it has been found to be favorable to use fabrics made of cellulosic fibers e.g., a hydro-entangled cotton web of 35 g/m², available from Winner Medical, China, under the trade designation PCO-35-N.

The absorbent core of the absorbent pad may comprise a pad acquisition/distribution layer (ADL) 2250, instead of or additionally to a chassis fluid handling aid material 1250, if present. Such an acquisition material is well known to a person skilled in the art, but should be adapted to the particular intended use.

The absorbent core further comprises absorbent material, preferably including superabsorbent polymer (SAP) material, as may be combined with fibrous material, e.g., cellulosic pulp.

The absorbent core may be formed in-situ by combining SAP powder with fibrous material, such as pulp or synthetic webs, preferably stabilized by adhesives, or thermofusion, such as ultrasonic bonding, as well known in the art.

In particular when intended and adapted to female users, it may be preferred that the absorbent core allows to maintain a natural pH in the vaginal region of around 5.5, by exhibiting a pH value of less than 6 or less than 5 or even less than 4 after loading the absorbent core with six fold of its dry weight with 0.9% NaCl solution and measuring the pH by pressing a pH indicator paper (MERCK 1.09527.0001) against the surface of the wetted absorbent core at the point where the NaCl solution has been applied to, e.g. by comprising particular pulp such as “Bliss™ pulp” of International Paper Co. USA. Maintaining a pH below 6 in the absorbent core is beneficial to mask odor based on ammonia or TMA (Tri-Methyl-Amin) typical for menstrual fluid and urine. Furthermore, the low pH inhibits the growth of bacteria similar to the protective acid mantle of the human skin. This allows to relinquish the use of biocides as commonly used in washable menstrual pants.

The absorbent core may be pre-formed such as available under the trade designation eCore from Glatfelter Falkenhagen GmbH, Germany, or MM150.SMT.CSN00.V03.F from Glatfelter Falkenhagen GmbH, Germany, or Gelok 15040-71 S/S of Gelok Int. Corp., Dunbridge, USA, or C1250956 NovaZorb® from Engineered Absorbent Materials (EAM), USA, or SuperCore SCP-200-TCF from McAirlaid's Vliesstoffe GmbH, Germany.

Alternatively, the absorbent core can be made in-line of fluff fibers or a blend for SAP (Super Absorbent Polymer) and fluff-pulp fibers formed in-line using hammer mills and drum formers on a converting line, or by combining SAP particles with synthetic fibers.

The absorbent core is preferably thin, preferably exhibiting a dry thickness of less than about 5 mm, or less than about 3 mm, or less than about 2 mm, but typically more than about 0.1 mm.

The absorbent core may exhibit an overall liquid retention capacity which does not need to be maximized, as the absorbent pad can be easily replaced without changing the chassis.

Thus, the overall liquid retention capacity should be more than about 10 ml, or more than about 20 ml, or more than about 50 ml, or more than about 100 ml, but typically less than about 500 ml, or less than about 300 ml, according to “Centrifuge Capacity” (modified MWSP 241.0) determination, as described below.

In addition, a high local basis capacity, expressed as absorbent capacity per unit area, should not be excessive as otherwise it may result in an undesired bulky wearing impression.

This may be achieved, if the absorbent core exhibits a basis capacity of more than about 2000 ml/m², or more than about 4000 ml/m², or more than about 7000 ml/m². but less than about 16000 ml/m², or less than about 13000 ml/m², or less than about 10000 ml/m², calculated from the above reference centrifuge capacity according to modified EDANA NWSP 241.0.

Optionally, the absorbent core may comprise odor masking or reducing agents, as may be perfumes, odor adsorbents, or bacterial growth inhibitors, such as—without limitation—based on zinc ricinoleate, e.g., Zinc ricinoleate Methylglycinediacetic acid, N,N-BIS(carboxylatomethyl)alanine trisodium salt (MGDA) as commercially available under the designation FRANCHEM® Sorb 30 B from Surfachem Deutschland GmbH;
comprising Cyclodextrin e.g., CAVAMAX® from Wacker Chemie AG, Germany;
comprising silver ions, as available in silver chloride, optionally as nanoparticles.

Optionally, an employed superabsorbent polymer may be modified for an enhanced odor control function, e.g., by comprising urease blocking functionality, such as provided by SAP type OC-301 supplied by LG-Chem, South Korea.

Optionally, a particularly modified cellulose fluff pulp may be utilized, such as exhibiting a pH of less than 6 or less than 5 or even less than 4, e.g., the above referenced “Bliss™ pulp of International Paper Co. USA or Supersoft® Odor Control Fluff Pulp from International Paper, USA.

The application of the absorbent pad varies from daily use, menstruation, light incontinence and heavy incontinence. Therefore, different test methods have to be applied for the determination of the acquisition and rewet depending on the target application, in particular relating to urine or menses.

Preferably, the absorbent core comprises absorbent material, optionally superabsorbent material such that it exhibits an absorbent capacity, as determined according to the centrifuge capacity test as described herein below, of more than 10 ml, preferably 50 ml or even 100 ml depending on the application (menstrual pant or incontinence pant). Preferably, the absorbent pad exhibits a rewet value of at less than 10 g, or less than 5 g, or less than about 1 g when submitted to the urine related acquisition and rewet test for an absorbent pad, as described herein below.

Preferably, the absorbent pad exhibits an acquisition value of at least less than 300 sec, when submitted to the urine related acquisition and rewet test for an absorbent pad, as described herein below.

In particular when intended to be used in the context of menstrual products, the absorbent core may exhibit an acquisition value of at least less than 20 sec, when submitted to the menses related acquisition and rewet test for an absorbent pad, as described herein below, or rewet value of less than about 5 g, when submitted to the menses related acquisition and rewet test for an absorbent pad.

The absorbent pad may further comprise an outer barrier 2900 preventing liquid penetration to the outside, e.g., the garments. Such a barrier is well known in the art, and may be a conventional lower core cover sheet 2920 for hygiene materials, such as film material, e.g., PE or PP films of typically less than about 25 g/m² or less than about 20 g/m², optionally in combination with a fibrous layer (also referred to as “textile backsheet” material), hydrophobic NW material, e.g., PP-SMS @18 g/m² or SSMMSS and the like. Alternatively, the outer layer 2900 may also be liquid permeable, as will be further described herein for designs with a chassis fluid handling aid material positioned on the outer portion of a pocket.

The upper core cover sheet and the lower core cover sheet of the absorbent pad envelope the absorbent core, which can be achieved by conventional designs. In a first execution, see FIG. 2D, the lower core cover sheet may be folded around the sides of the core overlapping the upper core cover sheet along the periphery of the absorbent pad. In a second execution, the lower core cover sheet may be folded around the sides of the core, but on the chassis-oriented side of the absorbent pad it is positioned underneath the upper core cover sheet, i.e., between the upper core cover sheet and the absorbent core, whereby the upper core cover sheet may also be folded around the side towards the lower core cover sheet side to provide a more pleasant look, although in this design the upper core cover sheet should not exhibit lateral wicking.

In a further execution, see FIG. 2C, the upper core cover sheet and the lower core cover sheet extend outwardly beyond the absorbent core and may be connected in the thus formed periphery in a “butt” mode, whereby the general inner surfaces of the webs are in a touching position.

The absorbent pad may be constructed by applying conventional technologies, such as gluing, and/or thermo-bonded, optionally ultrasonic bonding.

Preferably, the absorbent pad exhibits a balanced stiffness or bending resistance, as may ease the handling, in particular when inserting the absorbent pad into the pocket. In particular, for applications where the absorbent pad is to be positioned in the crotch region of a wearer, the absorbent pad should be perceived as soft, or supple, conforming readily to width-wise compression by the legs.

To this end, it may be preferred that the bending stiffness according to “Stiffness of Nonwoven Fabrics Using the Cantilever Test (INDA) NWSP 090.1.R0 (15) is
for the longitudinal direction less than about 200 mN*cm, or less than about 150 mN*cm, or less than about 80 mN*cm, but typically more than about 10 mN*cm, or more than about 20 mN*cm;
for the width direction less than about 150 mN*cm, or less than about 100 mN*cm, or less than about 60 mN*cm, but typically more than about 10 mN*cm, or more than about 20 mN*cm,
along the longitudinal direction higher by more than about 5%, or more than about 10%, or more than about 20% compared to and based on the one in the width direction.

This may be achieved by applying particular glue or embossing lines to the absorbent pad or its core, which may generally extend longitudinally but with a curved shape.

Optionally, the absorbent pad may comprise handling aids, such as a pouch 3400 on its outer surface, adapted and sized such that one or more fingers can be inserted for pushing the pad into its proper position.

Another optional handling aid for application of the absorbent pad may be a separate application aid exhibiting the required stiffness as described for the absorbent pad, but which is removed after the application and may be re-used for further applications in analogy to a shoehorn.

Optionally, for executions where absorbent pad comprises attachment elements on its surface that may have the tendency to adhere to surfaces prior to be positioned properly, such as adhesive stripes or mechanical fastener hooks, the upper core cover sheet surface of the absorbent pad may comprise a release paper, optionally comprising a removal strip allowing to pull the release paper away, once the pad is put in place.

Optionally the absorbent pad may comprise removal aids as a handling aid. In a first execution, when the upper core cover sheet and the lower core cover sheet are connected in a “butt” mode, whereby the general inner surfaces of the webs are in a touching arrangement, in the outer periphery of the pad, the removal aid may be an enlarged tab like portion of the periphery.

Alternatively, a tab may be connected to the pad, optionally as a loop or arched handle, adapted to finger pouch 3400 for easing application.

It is further a particular aspect of the present invention that the chassis and the absorbent pad as described in the above can be combined to a fully functional absorbent hygiene article system, whereby each of the two may be replaced independently from the other, whereby the absorbent pad is positioned such that the fluid receiving surface is oriented to and in good liquid contact with the intake zone of the chassis. “Good liquid contact” refers to an arrangement, wherein fluid may transfer from the intake zone of the chassis without significant reduction of the fluid transfer properties, when compared to an arrangement of connecting the two elements to each other under laboratory testing conditions combining the two layers by application of gentle pressure.

The user may don the well-fitting chassis, either with an absorbent pad already inserted or without that. Sometimes, applying the absorbent pad after donning of the chassis may be easier and the pad may be properly positioned upon application.

A further particular execution of a hygiene article system 100 is described by referring to FIG. 4. For this combination of the chassis 1000 and the absorbent pad 2000, the chassis comprises a pocket 3200 adapted to receive the absorbent pad 2000 with a pocket opening 3210, optionally reclosable, and a pocket attachment 3280 to the outer surface of the chassis. Further, the chassis 1000 comprises a chassis fluid handling aid material 1250. In a first execution, the chassis fluid handling aid material 1250 is connected to or integral with the materials of the intake zone 1220, e.g., positioned opposite of the inner surface layer 1100 of the chassis 1000, see FIG. 4A, allowing fluid to pass through towards the user-oriented surface 2100 of the underlying absorbent pad 2000.

In a second execution, see FIG. 4B, the chassis fluid handling aid material 1250 is connected to or integral with the pocket material 3220, opposite of the liquid impermeable outer surface 3290 of the pocket material, i.e., positioned towards the absorbent pad 2000, once inserted into the pocket and with all of the surface of the absorbent pad being liquid permeable. In a third execution, see FIG. 4C, the chassis comprises two chassis fluid handling aid material 1250′ and 1250″, positioned such that an absorbent pad 2000, with all of its surfaces being liquid permeable, can be sandwiched between these. For any of these executions, the absorbent pad 2000 comprises an absorbent core 2200 that is adapted to absorb liquid from the chassis fluid handling layer, such as by exhibiting a higher capillary suction, preferably further supported by osmotic suction of SAP comprised therein, preferably at a weight ratio of more than about 60% SAP in the absorbent core. A particularly suited execution for the chassis fluid handling aid material 1250 may be a high loft acquisition layer, such as available under the designation DRY WEB TDL2, at 60 g/m² from TWE GmbH & Co. KG, Germany. In a preferred execution, the dimensions of the absorbent pad are at least laterally smaller than the ones of the pocket, thereby easing the application and removal of the absorbent pad. In order to avoid contact of the user replacing a used core with the wet fluid handling aid 1250″ it is favorable to choose the fluid handling layers such that the layer 1250′ is more hydrophilic as layer 1250″ so that the layer 1250″ does not contain significant amount of liquid so far layer 1250′ and the core 2200 are not fully loaded.

Thus, the system according to the present invention provides particular fluid handling properties, in that it allows a fluid handling aid, as may be positioned in the liquid intake region of the article chassis to receive the liquid rapidly and store it by capillary force, but also release the liquid to an absorbent pad, even if this exhibits a smaller size than the chassis fluid handling aid material.

This can be demonstrated by determining the amount of liquid as being released from a material for a fluid handling aid into a smaller sized absorbent pad, according to the test for “Liquid release of fluid handling aid material to ultimate liquid storage material” as described herein below.

A suitable exemplary material for a fluid handling aid material is commercially available under the trade designation ATB Multifunctional Blue A49B60 from Texsus spa, Italy, at 60 g/m² basis weight. When combined with a 180 g/m² basis weight airlaid absorbent pad material from Glatfelter GmbH, Germany, code MM180.SMT.CSNOO.V03.F.PEFC.S.046w, the fluid handling aid material released 85.1% of the total fluid pick-up. Similarly, when combined with a 270 g/m² eCore absorbent pad material from Glatfelter Falkenhagen GmbH, Germany, code MM270.PTB.CSTOO.VOO.R.FSCM.2x0046w.1050d, the fluid handling aid material released 92.0% of the total fluid pick up.

This demonstrates that when removing the absorbent pad from the hygiene article chassis, e.g., for replacing it by a fresh absorbent pad, the chassis fluid handling aid material is dried out to a significant degree and ready to receive further fluid load for release to the fresh absorbent pad.

### Test Methods

All percentages are on a weight basis, unless expressly referred to otherwise.

All testing should be performed in a conditioned room maintained at about 23+/−2° C. and about 50+/−2% relative humidity.

All equipment should exhibit an accuracy of at least one more digit than the reporting of the resulting measurements.

If a material exhibits a sidedness and the test results are susceptible to this, the test should be executed according to the intended “in-use” orientation. “NWSP” refers to the “Nonwovens Standard Procedures”, Edition 2015 (suffix R0.15), issued by EDANA, Belgium.
“Repeated liquid strike through”—NWSP 070.7 & “wet-back after repeated strike through” (rewet) (NWSP 070.8) for nonwoven materials.
“Water Vapor Transmission Rate” (WVTR) by the principle of measuring the time to increase humidity (“Lyssy”)—Part 2, NWSP 070.6.
“Vertical Wicking Test”: A strip of sample material at a width of 50 mm is vertically hanged such that it is immersed 1 cm deep into a reservoir of 0.9% saline. The time is recorded after the sample is immersed and the corresponding weight change of the liquid in the reservoir is recorded. The reservoir is sufficiently large such that the change of fluid level is negligible. After 30 mins the Vertical Wicking Height has essentially reached a steady value, which is then recorded.
“Liquid Flow Resistance Test”: One end of a strip of test material at a width 50 mm and sufficient length is positioned horizontally into a reservoir of 0.9% saline as test liquid. The strip is further supported on a beam of 1 cm diameter, such as of metal or polymethylmethacrylate, positioned 5 cm above the liquid level of the reservoir. 10 cm of the free end of the strip hang freely vertically over a beaker for receiving transported fluid, positioned on a balance. Liquid is now first sucked against gravity towards the maximum height on the beam, and then released vertically downwardly. The time is recorded after the first drop is released. The amount of transferred liquid is monitored via the balance. Plotting the fluid uptake in gram versus time in minutes results in a straight line, and the slope thereof in gram/minute is the Fluid Transport Rate as a measure for the Liquid Flow Resistance.
“Horizontal Liquid Transfer Test”: One end of a strip of 50 mm width material is positioned horizontally into a reservoir with 0.9 w-% saline sol. The other end of the strip is positioned on a balance on top of a plastic plate of PMMA at a height level with the liquid level in the reservoir positioned laterally 2 cm away from the reservoir. The reservoir and the PMMA plate on the balance are horizontally levelled. The test material is laid at a height of 0.5 cm above the reservoir level over an intermediate round beam of 1 cm, such as of metal or polymethylmethacrylate. Below the strip, a standard absorbent material is placed on top of the test material. The standard absorbent material is made of an airlaid material available from Glatfelter, Germany, under the trade designation MM460.SMT.CST00.V01.J.NOCF, with
basis weight of 460 g/m² (NWSP 130.1.R0 (15),
60 w-% of SAP,
thickness 1.8 mm (NWSP 120.6.R0 (15),
calculated density of 0.256 g/m³;
dry tensile strength—MD) 48 N/50 mm (NWSP 10.1.R0 (15),
a “Liquid Retention” of 20 g/g,
all according to the respective tests as described herein below.

The airlaid material is cut to a width of 46 mm and further enveloped by a hydro-entangled cotton web of 35 g/m², available from Winner Medical, China, under the trade designation PCO-35-N, thus forming the standard absorbent material at a width of 50 mm.

A further plastic plate of 22 cm by 7 cm and a weight of 140 g is placed over the stripe and the standard absorbent material, and a further weight of 400 g is placed there on, such as to create a pressure of 344 N/m². The Liquid Uptake on the balance is monitored over the time, and reported as grams liquid at 5, 10, and 30 minutes.
“Vertical Liquid Transfer”: One end of a strip of test material of 50 mm width and 20 cm length is clamped flush together with a strip of standard absorbent material as employed for the “Horizontal Liquid Transfer Test”, an airlaid material available from Glatfelter, Germany, under the trade designation MM460.SMT.CSTOO.V01.J.NOCF, wrapped in a 35 g/m² hydro-entangled cotton, of 17 cm length between two plastic plates of PMMA (22 cm×7 cm×0.8 cm), which are standing vertically on a clamp and are connected to each other by adhesive tape at a distance of the combined thickness of the test material and the standard absorbent material, e.g., at about 5 mm. This set up is further clamped at the top such that the 3 cm of the test material strip extend over the plastic plates with the standard absorbent core, and at least 1 cm thereof is dipping into the test liquid of 0.9% saline in a reservoir, which is placed on a balance.

The liquid transfer over the time is monitored on the balance, and reported as grams liquid at 1, 3, and 15 minutes.
“Air permeability”: American Society for Testing and Materials (ASTM) D737-18 at 125 Pa and a surface of 20 cm².
“pH measurement of cellulose material”: Wetting the pulp/core pouring the six-fold of its own dry-weight of an aqueous 0.9% NaCl solution on the upper surface of the cellulose and after 5 minutes using a pH indicator paper (Merck 1.09527.0001 or equivalent) to determine the pH by pressing the indicator paper with about 0.02 bar against the wetted surface at the intake point of the cellulose.
“Free Swell Absorption Capacity: Following the Test Method PA07/05 of Hygiene-Technologie GmbH (Hy-Tec), Haan, Germany, the test specimen, as may be material of the intake zone or of the absorbent core, is submersed in sufficient 0.9 w-% saline for 30 mins, after which is removed and placed on a coarse grid or sieve, e.g., a grid with 1 cm wide opening and ribs of about 2 mm. After a further waiting time of 10 mins, the weight of the test specimen is recorded as free swell absorbency in grams.
“Liquid Retention—Centrifuge Capacity”: This test is generally following the EDANA NWSP 241.0.R2 (15) “Polyacrylate Superabsorbent Powders—Determination of the Fluid Retention Capacity in Saline Solution by Gravimetric Measurement Following Centrifugation,” but applying it to an absorbent material, as per Test Method PA07/05 of Hygiene-Technologie GmbH (Hy-Tec), Haan, Germany. To this end, a test sample as treated according to the “Free Swell Absorption Capacity” test is positioned within a commercial centrifuge exerting a centrifugal force by having an internal diameter of 225 mm at a rotation of 2800 rpm) for at least 1 min. The test specimen is removed and weighed and the centrifuge absorbent capacity recorded in grams.

For materials as being incorporated into products or articles, these are removed, such as by cutting, from the product or article.
“Liquid Intake Time And Rewet”

The Acquisition/Rewet measurement of products designed for use in Adult Incontinence Test Method PA07/03 of Hygiene-Technologie GmbH (Hy-Tec), Haan, Germany.

The test employs an apertured Perspex plate of 70 mm by 220 mm with a central cylinder of an inner diameter of 20 mm and a height of 130 mm at a weight of 255 g, adapted such that weights of 2 kg each can be placed on onto the plate on either side of the cylinder. Upon placing the cylinder and the plate with weights centered on the plate, 50 ml of 0.9 w-% saline as synthetic urine are applied to the cylinder, and the acquisition time until complete disappearance of the liquid is recorded as 1^{st} gush acquisition time. 20 mins after that point in time, the second 50 ml are applied, and the 2^{nd} gush acquisition time until disappearance is recorded. After another 20 mins, the 3^{rd} 50 ml are applied and the 3^{rd} acquisition time is recorded. After 5 mins, the weights, plate and cylinder are removed and accurately (i.e., +/−0.01 g or better) weighed 50 g of filter paper of 110 mm by 230 mm, are placed on the test specimen, followed directly by applying a plate without cylinder at 145 g and the 2*2 kg weights. After 15 secs, the weights and plate are removed and the rewetted filter paper is accurately weighed. The three acquisition times are added to the cumulative acquisition time in seconds, and the rewet value is recorded in grams.
“Menses related Acquisition and Rewet Determination—absorbent pads”

The Acquisition/Rewet measurement for the absorbent pads, which are applied during menstruation is based on the specification of PA 05/02 and PA 05/01 from Hygiene Technologie GmbH (Hy-Tec). The sample is placed horizontal on a plate. A ring out of Teflon (80±1 g, inner diameter 30±0.1 mm, height 21.5 mm) is positioned in the middle of the sample. Afterwards, the test fluid artificial menses (solution of demineralized water with 1.4% Tylose H 20 P2, 0.4% Sodium Hydrogen Carbonate, 0.5% Sodium Chloride, 0.025% Alura Red Dye, 10% glycerin) is applied from 1 cm above the ring into the ring. The time until the fluid is absorbed into the pad is measured (=acquisition). After 10 minutes, filter paper (type 604, approximately 10 sheets, 140×85 mm) is weighed and placed with a weight on top of the wetted sample as described above. After 15 s the weight and the filter paper sheets are removed. The filter paper sheets are weighed again. The increased mass of the filter paper sheets due to fluid uptake is calculated (=rewet).
“Surface pH measurement of absorbent pad”: Gently pressing a pH indicator paper (MERCK 1.09527.0001 or equivalent) at a pressure of about 0.02 bar against the surface of the of the absorbent pad intended to be directed towards the intake zone of the chassis after slowly pouring, avoiding liquid run-off, an amount of half of the free swell absorption capacity (Following the “Free Swell Absorption Capacity Test” Method, as described in the above) of the absorbent pad of an aqueous 0.9% NaCl solution onto the surface of the absorbent pad intended to be directed towards the intake zone of the chassis and waiting for 5 minutes previous to measuring the pH.
“Topsheet Run-off Test”: This test aims at determining the ability of the material to retain liquid applied thereto. This test is based on the EDANA test method Nonwoven Coverstock Run-Off test NWSP 080.9R0 (15), and employs a 10° inclined base plate onto which the test specimen was placed at a length of at least 25 cm extending from the lower end of the plate. At that point 25±0.5 g of 0.9 w % saline was applied through a centered tube with an inner diameter of 5 mm, distanced vertically 25 mm above the web, at a flow rate of 6.3 g/sec. After application of the fluid the weight increase of the web was recorded.
“Tensile properties”: The tensile properties of the materials may be determined according to the NWSP 110.4, for a material width of 25 mm, length 150 mm, a gage length of 75 mm, and an extension speed of 300 mm/min.
“Basis weight”: NWSP 130.1, adapted to respective materials.
“Single Load Drop Test”: At least three drops of de-ionized water/saline, for which contamination by any surface-active substance has been avoided, thus exhibiting a surface tension of at least 70 dyn/cm, are placed on a test specimen, such as by a clean pipette. By visual observation it is determined, if these drops are penetrating into the substrate or remain on the surface for at least 60 secs. In the latter case, the test specimen is considered hydrophobic, otherwise hydrophilic.
“Stiffness of Nonwoven Fabrics Using the Cantilever Test” (INDA) NWSP 090.1.
“Nonwoven Thickness” The thickness for the samples was determined generally following EDANA Standard Procedure NWSP 120.1, with a circular pressure foot with 3 cm diameter at a pressure of 15 g/m².
“Aperture measurements”: The geometric determination of the apertures and their patterns may be determined by any suitable optical method. A particular suitable method may be employed according to the disclosure in EP3215086, to which express reference is made for the aperture related aspects of the “Aperture/Feret Angle Test”, wherein aperture dimensions, effective open area and inter-aperture distance measurements are obtained from specimen images acquired using a flatbed scanner using a reflectance mode at a resolution of 6400 dpi and 8-bit grayscale. The scanner is interfaced with a computer running an image analysis program, and calibrated against an acquired image of a ruler certified by NIST. The test specimen may be mounted in a steel frame and be backed with a black glass tile.

1. A re-usable article chassis for being worn on the lower torso of a wearer exhibiting a user-oriented surface and an opposite outer surface,
said article chassis comprising
at least one liquid intake zone adapted to cover a region of the exudate releasing body openings, comprising chassis fluid handling aid material exhibiting, when tested on the surface adapted to be oriented towards the user,
a centrifuge capacity of
less than about 10.0 g/g
when tested according to the “Absorbent Capacity—Centrifuge Capacity” Test;
“Liquid Flow Rate” of more than about 0.4 g/min, when tested according to the “Liquid Flow Resistance Test”, as described herein;
a main chassis region
said article chassis further comprising at least one pocket covering at least a portion of said at least one intake zone, comprising a pocket opening adapted for receiving and positioning an absorbent pad in liquid contact with the liquid intake zone.

2. A re-usable article chassis according to claim 1, wherein said main chassis, said intake zone(s), consist essentially of materials and construction elements that are washable at at least 40° C.

3. A re-usable article chassis according to claim 1, wherein said pocket is accessible for a user in a pre-use configuration and in an in-use configuration.

4. A re-usable article chassis according to claim 1, further comprising one or more elements selected from the group consisting of
a) leg cuffs;
b) openable side closure elements that are in a closed configuration;
c) comprising materials exhibiting a Moisture Vapor Transmission Rate (MVTR) of at least 5000 g/m²/24 h, when tested according to “Water Vapor Transmission Rate by the Principle of Measuring the Time to Increase Humidity Lyssy/EDANA,” Part 2 NWSP 070.6.R0 (15);
d) comprising materials exhibiting an air permeability of at least 0.2 m³/(m²*s when tested according to ASTM D 737-18 measured at 125 Pa with a surface area of 20 cm²;
e) said main chassis region comprises material exhibiting a vertical capillary wicking height of less than 10 mm, according to AATCC 197, 2018 Edition 2018—Test Method for Vertical Wicking of Textiles;
f) a circumferential extensibility at least in a waist region of at least 10% of the unstretched circumference;
g) a circumferentially seamless material at least in the waist region of the chassis article;
h) said one or more intake zone(s) comprise(s) chassis fluid handling aid materials that satisfy one or more of the properties selected from the group consisting of:
h1) “Horizontal fluid transfer”
after 5 mins more than about 18 g;
after 10 mins more than about 30 g;
after 20 mins more than about 68 g;
h2) “Vertical fluid transfer”
after 1 min of more than about 4.0 g;
after 3 mins of more than about 8.0 g;
after 15 mins of more than about 18.0 g;
h3) “Vertical Wicking Height” of more than about 19 cm;
h4) “Vertical Fluid Uptake”
at 3 mins of more than about 2.0 g,
at 5 mins of more than about 6.0 g,
whereby all properties are described by parameter determined according to the tests as described herein;
i) said pocket comprises material at the surface opposite to the intake layer which is liquid impermeable;
j) the materials of the main chassis region and an intake region exhibit a difference of at least 10% of
repeated liquid strikethrough time (NWSP 070.7 R0 (15));
wetback after repeated strikethrough time (NWSP 070.8 R0 (15));
Vertical capillary wicking for 0.9% NaCl solution according to AATCC 197, 2018.

5. A hygiene article system, comprising a re-usable article chassis according to claim 1 and an absorbent pad,
said absorbent pad comprising
a upper core cover sheet with a wearer-oriented surface;
an opposite surface with a lower core cover sheet:
an absorbent core in between comprising absorbent pad absorbent material;
and said absorbent pad being adapted to be positioned in said pocket of said re-usable chassis in registry with at least a portion of said intake zone of said article chassis and said upper core cover sheet being oriented towards the user.

6. A hygiene article system according to claim 5, further comprising a handling aid for easing application and/or removal of said absorbent pad.

7. A hygiene article system according to claim 6, wherein said handling aid is a stiffening member, adapted to be used for multiple applications of absorbent pads.

8. A hygiene article system according to claim 6, wherein said handling aid is integral with said absorbent pad.

9. A hygiene article system according to claim 5, wherein said absorbent pad further comprises one or more of the elements selected from the group consisting of
m) a removable cover;
n) cellulosic pulp exhibiting a pH of less than 6, according to the “pH measurement test of cellulose”, as described herein;
o) an odor reducing, adsorbing or controlling agent;
p) an odor masking agent;
q) a bacterial growth inhibitor;
r) an inhibitor of the degradation of urea to ammonia and carbon dioxide;
s) absorbent material, such that said absorbent pad exhibits an absorbent capacity of more than 10 ml of an aqueous 0.9% NaCl solution;
t) absorbent material, said article exhibiting a rewet value of at less than 10 g, when submitted to the “Adult Incontinence Article: Rate of acquisition and Re-Wet test NWSP 070.9 R1 (15)”.

10. A hygiene article system according to claim 5, wherein said absorbent pad further exhibits one or more features selected from the group consisting of
u) a urine related Acquisition value of less than 35 sec, when submitted to the Urine related Acquisition and Rewet Determination Test PA07/03 (3×30 ml);
v) a urine related Rewet value of less than 15 g, when submitted to the Urine related Acquisition and Rewet Determination Test PA07/03;
w) a menses related Acquisition value of at least less than 20 sec when submitted to the Menses related Acquisition and Rewet Determination Test PA 05/02;
x) a menses related Rewet value of less than 5 g when submitted to the Menses related Acquisition and Rewet Determination Test PA 05/01;
y) a surface pH value of less than about 7.0, when tested according to the surface pH measurement test, as described herein.

11. A hygiene article system according to claim 5, wherein said absorbent pad exhibits a size smaller than said fluid intake zone of said article chassis.

12. A hygiene article system according to claim 5, wherein said hygiene article chassis comprises a chassis fluid handling aid material, positioned
in direct contact with said liquid intake zone of said article chassis, and/or
towards said opposite surface of said pocket of said article chassis, whereby said lower core cover sheet is liquid permeable.

13. A hygiene article system according to claim 12, wherein said hygiene article chassis comprises
a first chassis fluid handling aid material, positioned in direct contact with said user oriented liquid intake zone of said article chassis and a second chassis fluid handling aid material positioned towards said opposite surface of said pocket of said article chassis,
whereby said first chassis fluid handling aid material exhibits
a “Vertical Wicking Height” or
a “Vertical Fluid Uptake”
this is higher than the respective value for said second fluid handling aid, when tested according to the test described herein.

14. A re-usable article chassis according to claim 1, further comprising
a transition region connecting said intake zone and said main chassis region

15. re-usable article chassis according to claim 14, wherein said transition zone comprises material exhibiting a vertical capillary wicking height of less than 10 mm, according to AATCC 197, 2018 Edition, 2018—Test Method for Vertical Wicking of Textiles.

16. A hygiene article system according to claim 8, comprising one or more elements selected from the group consisting of
k) a tab or a handle as handling aid
k1) as an extension of a peripheral region of the pad; or
k2) attached to the outer surface of the pad;
l) a finger pouch on its outer surface as handling aid.
